(19) United States
(12) Patent Application Publication
(10) Pub. No.: US 2023/0337661 A1
(43) Pub. Date: Oct. 26, 2023

(54) METHOD FOR TREATING CANNABIS FLOWERS

(71) Applicant: URBAN AGRICULTURE MEIDLINGER ROMAN E.U., Wien (AT)

(72) Inventor: Roman MEIDLINGER, Vienna (AT)

(21) Appl. No.: 18/012,845

(22) PCT Filed: Jun. 29, 2021
(86) PCT No.: PCT/AT2021/060227

§ 371 Date: Dec. 23, 2022

(30) Foreign Application Priority Data
Jun. 30, 2020 (AT) A 50552/2020

(51) Int. Cl. A01N 3/02

(52) U.S. Cl. CPC A01N 3/02

(57) ABSTRACT

Summary

The invention relates to a method for treating Cannabis flowers, in which in a first step (3,4) at least one Cannabis flower is brought into contact with liquid nitrogen, in a second step (5) the at least one Cannabis flower is moved relative to an oxygen-containing gas, in particular air, and/or is exposed to an agitated oxygen-containing gas, and in a third step (7) the at least one Cannabis flower is freeze-dried.

The invention relates to a method for treating Cannabis flowers.

Cannabis flowers serve as the basis for various products, e.g. as a tobacco substitute. Cannabis has several advantages over traditional tobacco.

In order to be able to use Cannabis flowers, for example as a substitute for tobacco, the flowers have to be dried after harvesting. Drying is achieved according to the prior art by air drying in ambient air or also by freeze drying. Cannabis flowers harvested and dried using traditional methods have a brown or dark green hue after drying.

However, this coloring is undesirable for Cannabis flowers. While many plants are intended to be bright and bold in color, e.g. a strawberry should stay bright red and a pea bright green, it is the other way around for Cannabis flowers. Here it is considered a sign of quality if the flowers have a light, silvery hue. The green hue is due to the presence of chlorophyll in the flower. In order to create a silvery hue, the chlorophyll contained in the Cannabis flower must be altered so that the flower shines silvery due to the trichomes (plant hairs) that have grown on the flower, without being pushed into the background by a strong green tone.

It is therefore an object of the invention to provide a method for treating Cannabis flowers which changes the chlorophyll in the flower in such a way that the color intensity of the chlorophyll is reduced. Furthermore, the taste of the Cannabis flower should become more intense.

To solve this problem, a method according to the invention is provided, wherein in a first step at least one Cannabis flower is brought into contact with liquid nitrogen, in a second step the at least one Cannabis flower is moved relative to an oxygen-containing gas, in particular air, and/or is exposed to an agitated oxygen-containing gas, and in a third step the at least one Cannabis flower is freeze-dried. The Cannabis flower is first brought into contact with liquid nitrogen, then either agitated in a gas itself and/or exposed to an agitated gas (directly) and finally freeze-dried. Multiple Cannabis flowers are preferably subjected to the method according to the invention at the same time.

As has surprisingly turned out, this procedure significantly changes the chlorophyll in the Cannabis flower, so that the Cannabis flower is significantly lighter after the method is complete and, in particular, has a silvery sheen. By carrying out the method according to the invention, the color of the chlorophyll is changed and in particular the color intensity is reduced. In addition, the essential oils contained in the Cannabis flower are better preserved and the Cannabis flower tastes more intense.

Before the first step, the Cannabis flower is harvested, i.e. separated from the Cannabis plant. Provision is preferably made for the Cannabis flower to be harvested immediately before the first step. The time span between separating the flower from the stem of the plant and the start of the first step, i.e. bringing it into contact with liquid nitrogen, is preferably a maximum of 5 minutes, particularly preferably a maximum of 3 minutes.

Furthermore, it is preferably provided that the Cannabis flower is subjected directly to the first method step without intermediate treatment. In particular, the Cannabis flower is preferably not manicured prior to the first step. Such treatments of the flower are not necessary and would only degrade the quality of the end product.

In order to increase the efficiency of the method, it is further preferably provided that before the first step, several Cannabis flowers of a similar size are combined and the method is carried out with several flowers of the same size. The Cannabis flowers are thus sorted by size before they come into contact with the liquid nitrogen. This is advantageous because some parameters for the optimal implementation of the method according to the invention depend on the size of the treated Cannabis flower.

It is also preferably provided that the leaves of the Cannabis flower are not removed from the flower before the first method step, so that the Cannabis flower together with the leaves come into contact with liquid nitrogen in the first method step.

Before the first step, the Cannabis flowers are preferably at a temperature of approx. 15° to 30° Celsius, i.e. normal room temperature, for example.

Furthermore, it is preferably provided that the at least one Cannabis flower is moistened with a liquid, in particular with water, before the first step. Above all, this has a positive effect on the end result when already dried Cannabis flowers are subjected to the method according to the invention. Provision is preferably made for the at least one Cannabis flower to be harvested, dried and then moistened before the first method step.

It is preferably provided that the time interval between the moistening and the first step of the method, i.e. the bringing into contact with nitrogen, is a maximum of 30 seconds and preferably between 20 and 30 seconds. The at least one Cannabis flower is here preferably (in particular completely) immersed in a liquid bath or sprayed with a liquid. Provision is particularly preferably made for the Cannabis flower to be moistened essentially on all sides in order to bring about a uniform effect over the entire surface of the flower.

During the first step, the at least one Cannabis flower is brought into contact with liquid nitrogen, as a result of which the Cannabis flower(s) is or are shock-frozen. This process step can be carried out in different ways.

Provision is preferably made for the at least one Cannabis flower to be completely covered with nitrogen at least during a section during the first step. This exposes the entire surface of the flower to the nitrogen, creating an even effect throughout the flower.

In a preferred embodiment it is provided that the at least one Cannabis flower is immersed in a bath with liquid nitrogen in the first step. The Cannabis flower is here immersed in nitrogen. This is an easy and safe way to ensure all flowers are getting adequate exposure to the liquid nitrogen. Furthermore, with this procedure it is possible to precisely determine the duration of the contact of the Cannabis flower with the nitrogen and therefore to carry out a precisely defined procedure. Provision is preferably made for the Cannabis flower(s) to be placed in a bowl or on a tray, on which they can go directly into the nitrogen bath provided for this purpose and can then be removed from the nitrogen bath again.

Provision is preferably made for the Cannabis flower(s) to be guided through a nitrogen bath on a conveyor belt in the first step. For example, the Cannabis flowers are placed through a hopper onto a conveyor belt that is placed in sections in a nitrogen bath and guides the Cannabis flowers through and then out of the nitrogen bath. The flowers preferably fall into the nitrogen bath and onto the conveyor belt, which then transports the flowers out of the nitrogen bath again.

The nitrogen bath is preferably carried out in a specially designed shock freezer, which is explicitly designed for liquid nitrogen. For example, the Cannabis flowers go directly into the liquid nitrogen bath through an opening in the top center and are transported directly to the next processing step after shock freezing by means of a conveyor belt, the speed of which allows to determine the exact time the flowers remain in the liquid nitrogen bath.

In this case, it is preferably provided that the liquid nitrogen inside the shock freezer has a sufficiently high filling level in order to achieve complete coverage of all Cannabis flowers with the liquid nitrogen.

The optimal duration of the first step depends in particular on the size and type of flowers (due to the different density) and is therefore determined separately in each case. Basically, dense, large flowers need more time than airy, small flowers. Furthermore, the duration of the first step can also influence the final result, in particular the color of the flowers. The duration of the first step is preferably 60-120 seconds, particularly preferably 80-100 seconds.

In the second step, the at least one Cannabis flower is moved relative to an oxygen-containing gas, in particular air, or the oxygen-containing gas is moved relative to the Cannabis flower, so that a relative speed arises between the Cannabis flower and the gas due to the movement of the gas and/or the Cannabis flower. In this case, the Cannabis flower is surrounded by the gas and at least one Cannabis flower is moved and/or the gas is moved. For example, a fan can be used to aerate the Cannabis flower(s) accordingly, or the Cannabis flower is swiveled, e.g. rotated, manually or mechanically in the gas. Combinations of these are also possible. In the second step of the method, the relative speed between the Cannabis flower and the oxygen-containing gas is, for example, 1 to 4 m/s, preferably 2 to 3 m/s, particularly preferably approx. 2 m/s. It has been found that the method works particularly well at these relative speeds.

The gas used in the second step can be ordinary ambient air consisting essentially of nitrogen and oxygen. The gas preferably has an (air) humidity of 40-80%, preferably 50-70%, and/or a temperature of 15°-25° Celsius.

In order to prevent the flower(s) from unintentionally thawing during the second step, it is preferably provided that the gas which comes into contact with the Cannabis flower during the second step has a maximum temperature of 20° Celsius.

In order to further increase the efficiency of the method, it is preferably provided that leaves are additionally removed from the flower during the second step. This combines exposing the flower to the gas and removing the leaves, eliminating the need for separate leaf removal.

Furthermore, it is preferably provided that the second step lasts a maximum of 20 seconds, preferably a maximum of 15 seconds. The duration of the aeration of the flowers is therefore a maximum of 15 to 20 seconds. In order to achieve a particularly good effect, the duration of the second step is 12 to 18 seconds. This ensures, on the one hand, that the flowers come into contact with the gas for a sufficiently long period of time and, on the other hand, do not thaw.

In order to ensure the aeration of the Cannabis flowers during the second step, it is preferably provided that the at least one Cannabis flower rotates, preferably in a drum, during the second step. The rotation moves the Cannabis flower relative to the gas. Preferably, the Cannabis flowers are rotated in a drum designed to remove the leaves from the Cannabis flowers during rotation. The drum is particularly preferably part of a dry trimmer.

Provision is preferably made for the Cannabis flower to be cooled between the second and the third step. This ensures that the Cannabis flower does not unintentionally thaw between the second and the third step. Furthermore, the possible period of time between the second and the third step can be designed more flexibly and the third method step does not have to take place directly after the second step. The Cannabis flower(s) is/are preferably stored at an ambient temperature of approx. minus 25° Celsius.

In the third step, the at least one Cannabis flower is freeze-dried. Freeze drying is a common process in which the products to be dried are dried as gently as possible. The freeze-drying should preserve the current status as far as possible, i.e. the color and volume achieved by shock freezing. This is achieved by the fact that under negative pressure the aggregate state of water stored in the flowers passes from the solid state directly to the gaseous state, i.e. without the liquid intermediate step (sublimation). This preserves the previously achieved “silver effect” and transforms the Cannabis flower into a much higher quality product.

The invention further relates to a treated Cannabis flower obtainable by a method according to the invention.

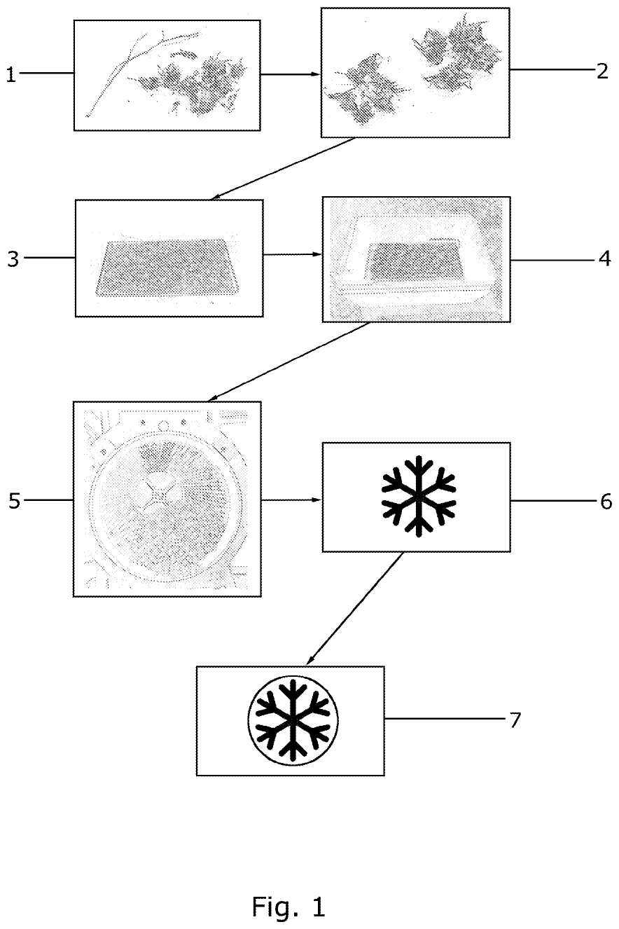

The invention is explained in more detail below with reference to an exemplary embodiment shown schematically in the drawing. FIG. 1 shows a flow chart of a method according to the invention.

In FIG. 1 an embodiment of the method according to the invention is shown schematically. First, the Cannabis flowers are harvested at 1 before the actual treatment process is carried out. Then, optionally at 2, the Cannabis flowers are sorted by size. The flowers are not (yet) manicured or the leaves removed. At 3 and 4 the first step of the method is carried out. The Cannabis flowers are placed on a tray and then placed with the tray in a shock freezer filled with liquid nitrogen so that all of the Cannabis flowers are completely immersed in the nitrogen. The Cannabis flowers are then removed from the nitrogen bath and, in a second step at 5, placed in a rotating drum of a dry trimmer. The dry trimmer rotates and moves the Cannabis flowers relative to the surrounding air, so that they are well aerated. In addition, the leaves are removed from the Cannabis flowers in the dry trimmer. After completing this second step, the Cannabis flowers are optionally cooled at 6. Finally, the Cannabis flowers are freeze-dried during the third step at 7. After freeze-drying, the Cannabis flowers can be further processed or packaged.

1. A method for treating Cannabis flowers, wherein at first in a first step (3,4) at least one Cannabis flower is brought into contact with liquid nitrogen, subsequently in a second step (5) the at least one Cannabis flower is moved relative to an oxygen-containing gas, in particular air, and/or is exposed to an agitated oxygen-containing gas, wherein the Cannabis flower is surrounded by the oxygen-containing gas, and finally in a third step (7) the at least one Cannabis flower is freeze-dried.

2. The method according to claim 1, wherein the at least one Cannabis flower is harvested immediately before the first step (3,4).

3. The method according to claim 1, wherein the at least one Cannabis flower is immersed in a bath with liquid nitrogen in the first step (3,4).

4. The method according to claim 1, wherein the at least one Cannabis flower is guided through a nitrogen bath on a conveyor belt in the first step (3, 4).

5. The method according to claim 1, wherein the duration of the first step (3,4) is 60-120 seconds, particularly preferably 80-100 seconds.

6. The method according to claim 1, wherein the relative speed between the Cannabis flower and the oxygen-containing gas in the second step (5) of the method is 2 to 3 m/s.

7. The method according to claim 1, wherein the oxygen-containing gas used in the second step (5) has a humidity of 40-80%, preferably 50-70%.

8. The method according to claim 1, wherein the gas which comes into contact with the at least one Cannabis flower during the second step (5) has a maximum temperature of 20° C.

9. The method according to claim 1, wherein leaves are removed from the at least one Cannabis flower during the second step (5).

10. The method according to claim 1, wherein the second step (5) is a maximum of 20 seconds, preferably a maximum of 15 seconds, long.

11. The method according to claim 1, wherein the at least one Cannabis flower rotates in a drum during the second step (5).

12. The method according to claim 1, wherein the at least one Cannabis flower is cooled between the second (5) and the third (7) step.

13. Treated Cannabis flower obtainable by a method according to claim 1.
